(19) United States
(12) Patent Application Publication
(10) Pub. No.: US 2009/0118838 A1
(43) Pub. Date: May 7, 2009

(54) Materials Useful for Support and/or Replacement of Tissue and the Use Thereof for Making Prostheses

(76) Inventors: Christian Debry, Strasbourg (FR); Philippe Schultz, Oberschaeffolsheim (FR); Andre Walder, L'Hay-Les-Roses (FR); Jean-Claude Voegel, Valff (FR); Dominique Vautier, Wisches (FR)

Correspondence Address: NIXON & VANDERHYE, PC, 901 NORTH GLEBE ROAD, 11TH FLOOR, ARLINGTON, VA, 22203, US

(21) Appl. No.: 11/989,941

(22) PCT Filed: Aug. 5, 2005
(86) PCT No.: PCT/EP2005/009228

§ 371 Date: Apr. 21, 2008

(51) Int. Cl. A61F 2/02; A61F 2/28

(52) U.S. Cl. 623/23.72; 623/16.11

(57) ABSTRACT

The invention relates to a material providing support and/or replacement of living tissues and the use thereof for manufacturing prostheses. The material according to the invention comprises microparticles of a biomaterial coated with polyelectrolyte multilayers containing one or more biologically active products.

The invention relates to materials providing a support and/or replacement of tissue and the use thereof for manufacturing prostheses.

The invention particularly relates to a material useful for making tracheal or laryngeal prostheses.

Total laryngectomy is the surgical procedure used to treat patients with advanced-stage cancer of the larynx. One major consequence of the treatment is a permanent loss of voice. Furthermore, respiration is definitively separated from deglutition, necessitating a permanent breathing opening in the neck. To date, artificial larynx reconstruction faces difficulties to comply simultaneously with the combined constraints of biocompatibility and restoration of the function.

To improve the biocompatibility, of implanted prostheses, one approach consists in the development of bioinert materials and, through surface modifications, create a bioactive interface that could regulate biological responses in a controlled way using specific cell signaling molecules or adhesion ligands. EP 856 299 B1 document thus relates to metallic prosthesis made of titanium beads for the support end/or replacement of open cell tissue, in particular for cervico-maxillo-facial implantation, especially for laryngeal reconstruction.

Recently, a new approach of tunable surfaces has been proposed to prepare biologically active surfaces. It consists in the alternate layer-by-layer deposition of polycations and polyanions for the build-up of multilayered polyelectrolyte films. The method is versatile, yet simple and applicable for materials of any type, size, or shape (including implants with complex geometries and textures, e.g., stents and crimped blood vessel prostheses).

Biomaterials comprising a core coated with alternative layers of polyelectrolytes with opposite charges, which serve as anchoring means for fixing biologically active molecules are disclosed, for example, in FR 2 823 675.

Adhesion of chondrosarcoma cells on a three-dimensional environment made of titanium beads modified by PLL, PGA or poly (sodium 4-styrenesulfonate) (PSS) ending multilayers was investigated. 3-D titanium surface covered by films terminating with negatively charged PGA or PSS amplified the occurrence and length of cell protrusions, whereas positively PLL charged surface down-regulate both β-tubulin and phosphorylated p44/42 MAPK/ERK expressions. These preliminary 15, data showed the potentiality of polyelectrolyte multilayer implant coatings to modify contractile and protrusive contact-based chondrocyte adhesion (Vautier et al., Cell Motil Cytoskeleton 2003, 56: 147-58).

Applications in the biomedical field are however still scarce due difficulties which are specific to in vivo conditions and traumatisms resulting from surgery. After implantation, biomaterials are spontaneously covered by a layer of host proteins followed by inflammatory cell attraction which may lead to degradative activities on the implant surfaces, resulting in complications, ultimately leading to the rejection of the prosthesis.

Particularly, the host proteins are present at high concentrations and will be absorbed on the implant surfaces, impeding contacts between cells and bioactive products adsorbed on or embedded in implant surfaces.

Other difficulties encountered when working under in vivo conditions are due to the septic environment which allow inflammatory responses. Moreover, presence of blood and various fragments can also damage the surface implants and the monocytes which are present on the implants are likely to remove active substances from the implant surfaces.

The inventors have surprisingly found that materials with a specific architecture were particularly useful as supports and/or replacements of living tissues. Experiments carried out in vivo with prosthesis manufactured with such materials have shown that they were not damaged in spite of the drastic environment and maintain the biological activity of the bioactive layers over a long period of time.

An object of the invention is then to provide materials whose architecture and composition are suitable for manufacturing prostheses for non temporary implantation in human or animals.

Another object of the invention is to provide prostheses for substitution of tissues, particularly of bones and/or cartilages and/or soft tissues.

The invention thus relates to materials useful for making support and/or replacement of living tissues, comprising microparticles of a biomaterial coated with polyelectrolyte multilayers containing one or more biologically active products wherein the microparticles have a particle size distribution between 50-800 μm, preferably 50-500 μm and are fused, the porous space between contiguous particles having an average dimension of 15 to 250 μm, preferably 15 to 150 μm.

As shown by in vivo experiments, such materials coated with biocompatible self-assembled layers are particularly valuable for making prostheses as they promote the biological effects of the active molecules bound to the layers.

Accordingly, the invention also relates to artificial prostheses for substitution of bones and/or cartilages and/or soft tissues.

The biologically active molecule(s) is (are) adsorbed on a polyelectrolyte multilayer film or alternatively is (are) embedded in a polyelectrolyte multilayer film.

The biologically active products, which are identical or different, can be at different depths in the multilayer architecture.

Suitable, biologically active products comprise peptides, polypeptides, amino-acids derivatives, growth factors, stem cells or drugs.

Antibiotics and particularly anti-inflammatory drugs will be used in the multilayers architecture. Appropriate anti-inflammatory drugs comprise α-melanocyte—stimulating hormone and/or its two analogues CP1 and CP2.

Unexpectedly, the implantation of such prostheses under the above mentioned drastic conditions, particularly in a septic environment does not result in an inflammatory response.

A particularly suitable prosthesis comprises polyelectrolyte multilayer films made of polypeptides selected in the group comprising poly (L-lysine) (PLL) and poly (L-glutamic acid) (PGA).

Advantageously, the α-MSH peptide is covalently bound to PGA adsorbed on or embedded in a polyelectrolyte multilayer film (PLL/PGA)₄.

Said microparticles are preferably made of titanium or a titanium-based alloy which may contain at least one other metal chosen from among indium, tin, niobium, palladium, zirconium, tantalum, chromium, gold and silicon.

According to an embodiment of the invention, the above defined prostheses are intended for substitution of tracheal or laryngeal cartilages, and have sizes corresponding to mean values of trachea or larynx diameter and length, with holes at both extremities and, if necessary, a longitudinal slot.

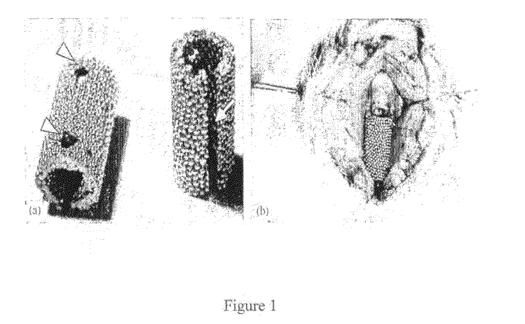

Other characteristics and advantages of the invention will be given hereinafter and comprise references to FIGS. 1 to 9, which respectively represent:

FIG. 1: a tracheal prosthesis according to the invention with holes at both extremities (arrow head) and a longitudinal slot (arrow) (FIG. 1A) and a prosthesis positioned in a rat tracheal (FIG. 1B);

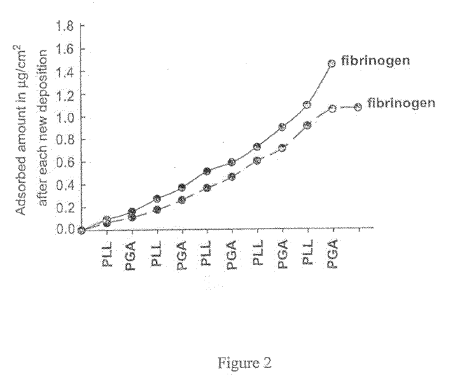

FIG. 2: the evolution of the increase in total mass of a PLL/PGA film after newly deposited polyelectrolytes and deposited fibrinogen found on the top of the film;

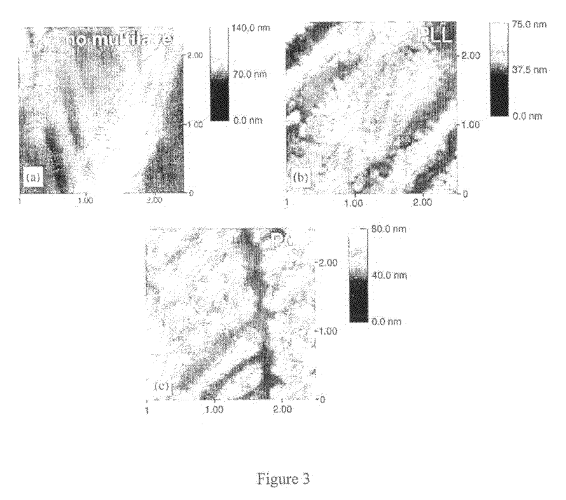

FIG. 3: AFM images of an untreated prosthesis (FIG. 3a) and of prostheses according to the invention (FIGS. 3b and 3c);

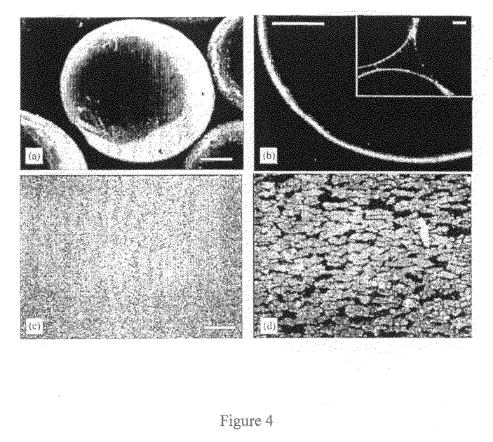

FIG. 4: the persistence of the multilayer films on titanium bead observed by SEM (FIG. 4a), on titanium beads according to the invention observed by CLSM (FIG. 4b); on silicone membrane before implantation (FIG. 4c); and 7 days after implantation in the trachea (FIG. 4d);

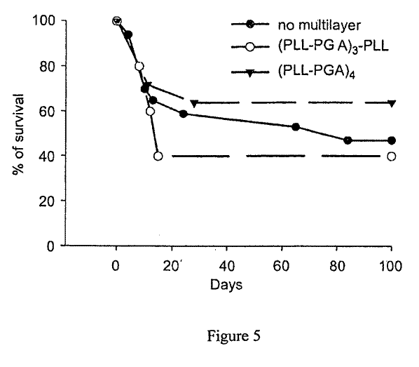

FIG. 5: the percentage of rat survival over a 100-day period after implantation with untreated prosthesis and prostheses according to the invention;

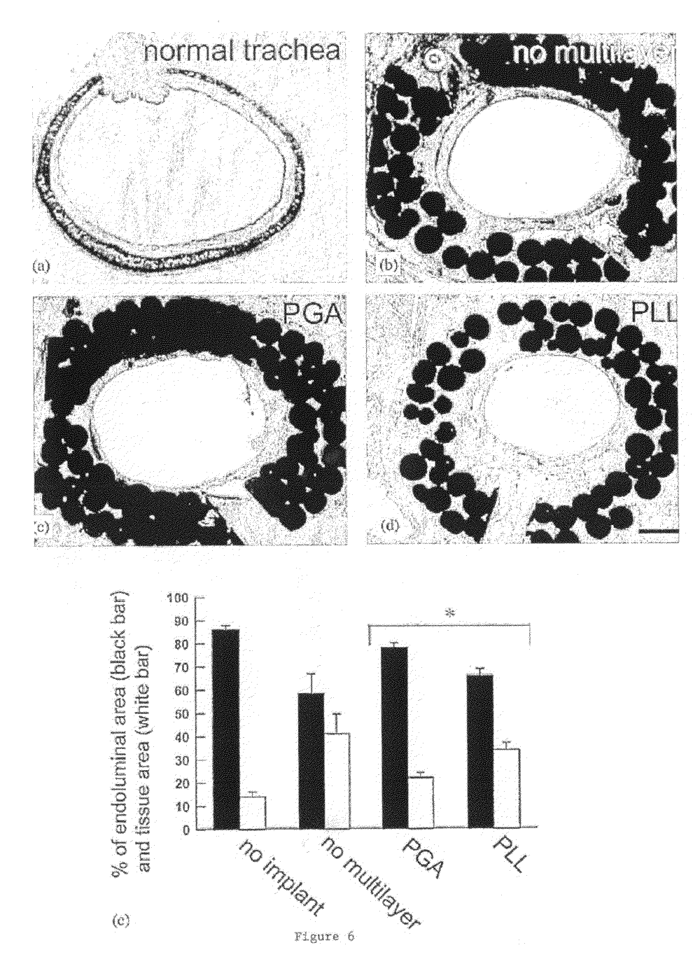

FIG. 6: photos of the transverse section of the cervical region 1 month after implantation;

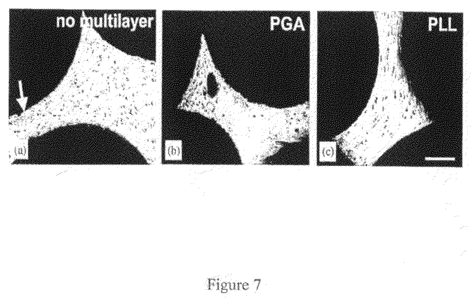

FIG. 7: photos of the transverse section of tracheal prosthesis showing details of the titanium porosity 1 month after implantation for untreated prosthesis (FIG. 7a) and prostheses according to the invention (FIGS. 7b and 7c);

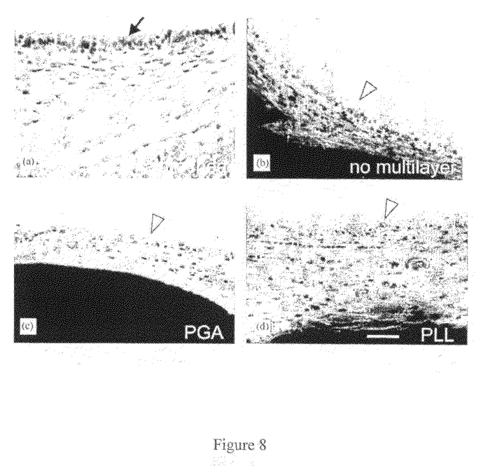

FIG. 8: photos of the transverse section 1 month after implantation showing details of the endoluminal side of: (a) section of rat trachea (normal trachea); (b) section of untreated titanium prosthesis (no multilayer); (c) section of titanium prostheses according to the invention; and

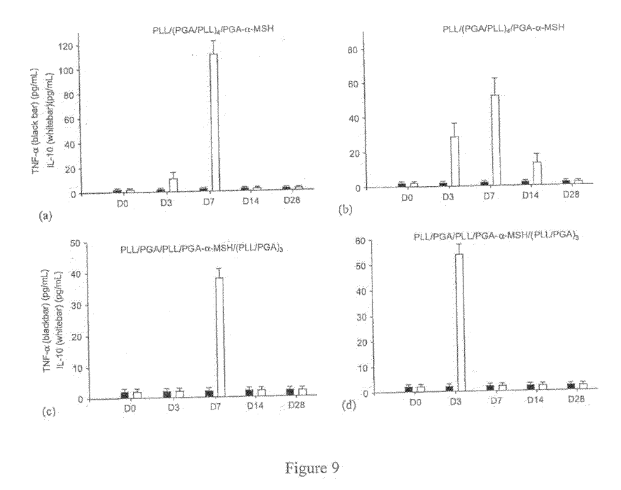

FIG. 9: results with different prostheses concerning systemic level of rat TNF-α and IL-10 secretion quantitated by ELISA, and corresponding to different implanted periods.

### EXPERIMENTAL

### Preparation of the Prostheses

The prostheses were made of spherical titanium beads of 400-500 μm diameter. Titanium used for these surgical implants was in conformity with the Association Française de NORmalization standards.

The beads were placed into a mold and were joined in order to obtain a self-supported part. The porous space between contiguous beads was about 150 μm. The prostheses sizes were adjusted on the mean values of trachea diameter and length previously determined from identical rats in age and weight to those used for in vivo experimentation. The prostheses consisted of cylindrical tubes of 10 mm length corresponding to six tracheal rings with an external diameter of 5 mm and an internal diameter of 3 mm.

On the prosthesis, a slot of 0.8 mm was incised into the tubes and a hole of 1 mm diameter was created at each extremity (FIG. 1a). The pieces obtained were tested for mechanical shock resistance. Before implantation, titanium prostheses were sterilized under ultraviolet light irradiation (254 nm) for 1 h.

### Polyelectrolytes and Solutions

PLL (MW 23.4×10³, Sigma, St. Louis, Mo.), PLL^{FITC} (MW 50.2×10³ Sigma, St. Louis, Mo.) and PGA (MW 54.8×10³, Sigma, St. Louis, Mo.) were used without any further purification. PLL, FITC PLL and PGA solutions were prepared at 1 mg/ml in 0.15 M NaCl.

Films were either built on titanium prostheses or silicon membranes deposited in 24-well plastic plates (NUNC).

Each sample was dipped for 20 min alternatively in 2 ml of the appropriate polycationic and polyanionic solution.

Each polyelectrolyte adsorption was followed by three rinsings of 5 min in 0.15 M NaCl solution.

At the end of the procedure, samples were sterilized for 15 min by UV (254 nm), stored at 4° C. and used within 1 week. Fibrinogen (Sigma, St. Louis, Mo.) was dissolved in 0.15 M NaCl at 7 μg/ml.

### Coupling of α-MSH to PGA

The α-MSH analogue CP2, of sequence SEQ ID No 1: HS-CH₂CH₂-CO-Ser-Tyr-Ser-Nle-Glu-His-D-Phe-Arg-Tryp-Gly-Ly-s-Pro-Val-NH₂, purified by high-performance liquid chromatography, was obtained from Neosystem (Strasbourg, France). Its coupling to PGA was processed through thiol-functionalization of PGA. PGA was conjugated to maleimide groups then mixed with peptide.

### Polyelectrolyte Multilayer Architectures

The following five architectures were built-up on titanium prostheses.

(1) (PLL/PGA)₃-PLL, (2) (PLL/PGA)₄, (3) (PLL/PGA)₄-PLL^{FITC}, (4) PLL/(PGA/PLL)₄/PGA-α-MSH and (5) PLL/PGA/PLL/PGA-α-MSH/(PLL/PGA)₃. Functionalized architectures were obtained by addition of PGA-α-MSH either on the top of the film (architecture 4) or under three (PLL/PGA) layer pairs (architecture 5).

### Surface Analysis

Optical Waveguide Lightmode Spectroscopy (OWLS)

Film growth was checked in situ by OWLS using TiO₂-coated waveguides (Microvacuum, Hungary).

The successive polyelectrolyte adsorptions were followed step by step according to the previously described procedure [32].

Atomic force images were obtained in contact mode in air with the Multimode Nanoscope VI from VEECO (Santa Barbara, Calif.).

Cantilevers with a spring constant of 0.03 N/m and silicon nitride tips were used (model MLCT-AUHW Park Scientific, Sunnyvale, Calif.).

Several scans were performed over a given surface area. The scans produced, reproducible images to ascertain that no sample damage was induced by the tip and that the observations were valid over large surface domains.

Areas of about 2.5 μm² were scanned with a scan rate between 2 and 4 Hz with a resolution of 512×512 pixels. A box of 0.5×0.5 μm² was displaced five times on the image and mean roughness (Rₐ) was calculated.

### Scanning Electron Microscopy (SEM) and CLSM

Titanium prostheses were mounted on sample holders with silver print, sputter-coated with a gold-palladium alloy in a Hummer J R (SIEMENS, Karlsruhe, Germany) unit and visualized by SEM with a JEOL JSM 35C (Tokyo, Japan) operating at 25 kV. CLSM observations were carried out on a Zeiss LSM 510 microscope.

FITC fluorescence was detected after excitation at 488 nm, cutoff dichroic mirror 488 nm, and emission band pass filter 505-530 nm (green).

Observations were done by using a x 63/1.4 oil immersion object.

### In Vivo Experiments

All animals were housed and fed in compliance with the “Guide for the Care and Use of Laboratory Animals” published by the National Institute of Health (NIH publication 85-23, revised 1985). Male Wistar rats (5-7 months old, 450-600 g) received an intraperitoneal injection of an anesthetic solution, 1/5 of 2% (vol/vol) Rompun (xylasin chlorhydrate 2% and methyl parabenzoate 0.1%) and 4/5 of 0.5% (vol/vol) Imalgen 1000 (pure ketamine).

A vertical median cervicotomy was performed from the sternum up to the jaw. Subhyoid muscles and nerves were separated from the trachea.

The trachea was incised between the second to the eighth tracheal cartilage.

To prevent retraction of the tracheal extremities a thin 10, band of tracheal membrane was conserved.

The longitudinal slot of the prosthesis was introduced into the posterior trachea. The tube was rotated in order to place the slot in a lateral position maintaining the extremities of the prosthesis in the aerial axis (FIG. 1b).

Thus, the posterior tracheal membranous band placed into the prosthesis lumen contributes to its positional stability.

The extremities of the prosthesis were then joined by Prolene 6-0 sutures (Johnson and Johnson thread) with tracheal tips, using the holes in the prosthesis.

Finally, subhyoid muscles were repositioned to cover the trachea and sutured back together.

The animals were subsequently housed in a controlled environment with 12-h light cycles. Food and water were provided ad libitum.

### Histologic Analysis

One month after prostheses implantation, rats were sacrificed by intra-peritoneal injection of a lethal dose of phenobarbital.

A large exaeresis was performed engulfing the cervical region into a single block to avoid mechanical separation of the prosthesis-tissue interface.

The specimens were fixed, dehydrated, and immersed into three successive methylmetacrylate baths containing increasing concentrations of catalysing agent.

The last bath was placed at 37° C. until full polymerization was completed; 200-μm thick sections were prepared using a LEICA 1600 microtome, and stained with Stevenel's blue-de Van Gieson's picrofuchsin for microscopic analysis.

The lumen and endoluminal tissue areas were manually delimited and measured using the software Lucia 2 (LW LUG-LUCIA 2, Nikon; Japan).

### Cytokine Measurements

At time periods (days 0, 3, 7, 14 and 28) after implantation of the prostheses, blood samples were collected from rat caudal artery in unheparinized tubes.

Blood samples were left at room temperature for 2 h to clot before centrifuging for 20 min at 2000×g.

Isolated sera were frozen and kept at −20° C. until assayed for the cytokine levels using ELISA specific for TNF-A and IL-10 (Quantikine, R&D Systems, Minneapolis, Minn.).

The secretion of TNF-α is an indicator of inflammation, whereas IL-10 secretion is an indicator of anti-inflammatory response and its induction is a positive response. Serial dilutions were performed to determine cytokine concentrations by comparison with the standard according to the manufacturer's instructions.

### Results and Discussion

### OWLS Analysis

In the case of blood-contact biomaterials, adsorbed fibrinogen is the primary component of plasma responsible for acute inflammatory response to implanted material. Thus, fibrinogen deposition was investigated on top of (PLL/PGA)3-PLL and (PLL/PGA)₄ films.

All the experiments were performed at the physiological pH of 7.4. The results are given on FIG. 2. The film characteristics were determined by OWLS without drying after each new polyelectrolyte deposition (full line: fibrinogen deposition on top of PLL ending film, dotted line: fibrinogen deposition on top of PGA ending film). The polyelectrolytes indicated on the abscissa scale (PLL or PGA) represent the last deposited polyelectrolyte.

Fibrinogen adsorption was important on (PLL/PGA)₃-PLL (FIG. 2, full line, 0.35 μg/cm²) but was strongly reduced on (PLL/PGA)₄ (FIG. 2, dotted line, 0.01 μg/cm²) as it was already observed for fetal bovine serum deposition on similar films. It was proposed that the adsorption of proteins from serum, which are mostly negatively charged at pH 7.4, is mainly driven by electrostatic forces.

In term of fibrinogen adsorption mediating acute inflammatory responses to implanted biomaterials, the results demonstrate that a (PLL/PGA)₄ multilayered film constitutes a more favourable coating for the prostheses.

### AFM Analysis

The surface topography of the polyelectrolyte multilayer film was also assessed using AFM.

The results are given on FIG. 3. Z scales are 140 nm for images (a), 75 nm for image (b), and 80 nm for image (c).

The naked titanium bead surface (no multilayer) displays a slightly striated topography (FIG. 3a). These surfaces features were partially masked by nanosized polyelectrolyte clusters reaching an average diameter of 280 nm for both coated multilayer surfaces (FIG. 3b: (PLL/PGA)₃-PLL) and FIG. 3c: (PLL/PGA)₄ films).

The surface roughness was quantified by the mean roughness value (Rₐ). For both (PLL/PGA)₃-PLL and (PLL/PGA)₄ films the Rₐ was equal to 4.2 nm, underlining the homogeneity of polyelectrolyte clusters distribution over surfaces.

CLSM Analysis

A fluorescently labeled PLL sample, PLL^{FITC} was used to monitor the deposition of the multilayered film on the titanium prosthesis (a detailed picture of a titanium bead by SEM is given in FIG. 4a) (bar=100 μm).

Fluorescence confocal microscopy observation of (PLL/PGA)₄-PLL^{FITC}-coated prosthesis confirmed the uniform pre-sence of PLL^{FITC} on this surface (FIG. 4b, green fluorescent continuous band around the bead) (bar=100 μm).

As it was previously demonstrated, PLL^{FITC} introduced at the outermost layer of the film diffuses through the whole film down to the substrate. Inset (FIG. 4b); (bar=85 μm) shows another focal plan with fluorescent bands surrounding three contiguous titanium beads.

To image the entire titanium bead covered by the fluorescent film, consecutive z-sections were collected at 4 μm intervals.

The continuous fluorescent band was present on all focal plans over the totality of the 500 μm diameter bead.

Since it was very difficult to observe the fluorescent film on a 3D surface, a plane silicon membrane coated with the same fluorescent film was used to evaluate the in vivo stability of the multilayer.

Thus, the coated silicone membrane was introduced into rat trachea lumen. Before implantation, the uniformly labeled surface (FIG. 4c) was observed.

Seven days after implantation, small areas without fluorescence were found due to a local film degradation and a possible peeling effect after explanation of the silicon membrane (FIG. 4d).

A local film degradation was previously found in vitro for (PGA-PLL)₁₉-PGA-PLL^{FITC} after 180 min of contact with THP-1 cells and a pronounced degradation after overnight contact with THP-1 cells was observed. Although the film contains less polyelectrolyte layers, it seems better conserved over a long period of time.

### Animal Implantation

### Animal Survival

Subsequent to prosthesis implantation, 50% (8 of 17 rats), 60% (7 of 11 rats) and 40% (2 of 5 rats) of rats implanted, respectively, with untreated prostheses and treated (PLL/PGA) 4 and (PLL/PGA)₃-PLL prostheses, survived over a period more than 100 days.

A strong mortality up to 20 days after implantation was observed, and a stable rate of animal survival after this period for either untreated or treated implanted prostheses used. The percentage of rat survival over a 100-day period is given on FIG. 5: Rat implanted with untreated prosthesis (no multilayer: filed circle), rat implanted with prosthesis modified by (PLL/PGA)₃-PLL multilayers abbreviated PLL (open circle), rat implanted with prosthesis modified by (PLL/PGA)₄ abbreviated PGA (filled triangle).

Here, compared to untreated prostheses, the prostheses coatings did not negatively influence the animal survival.

### Histological Analysis

One month after implantation, a global section of the cervical region was performed.

FIG. 6 gives a section of a trachea, without prosthesis (6a), with an uncoated prosthesis (6b), with (PLL/PGA)₄ (6c) and (PLL/PGA)₃-PLL (6d)-coated prostheses, respectively.

Among implanted prostheses (including untreated and prostheses coated with (PLL/PGA)₄ or (PLL/PGA)₃-PLL) films), prostheses treated with (PLL/PGA)₄ showed lumen areas close to the rat trachea areas.

The lumen area of (PLL/PGA)₄-coated prostheses increased significantly (by 12%) in comparison to (PLL/PGA)₃-PLL-coated prostheses (FIG. 6e), by respectively, 78±2% and 66±2.9%, 100%=lumen area (black bar) plus endoluminal tissue area (white bar) in the group “rat trachea”. A more regular and less obstructive endoluminal cell layer for prostheses treated with (PLL/PGA)₄ (FIG. 6c) was observed compared to prostheses treated with (PLL/PGA)₃-PLL (FIG. 6d).

This result shows that endoluminal tissue area depended on the multilayer polyelectrolyte ending layer.

A comparable cell colonization constituted by fibrous tissue and fibroblasts around the prostheses and within the empty spaces between the titanium beads was observed for the untreated prosthesis (FIG. 7a) as well as for (PLL/PGA)₄ (FIG. 7b) or (PLL/PGA)₃-PLL (FIG. 7c)-coated surfaces. Tissue present within the titanium porosity was well vascularized, as seen by the blood vessels found in the histologic section (FIG. 7a, arrow).

On the tracheal lumen side, a typical fibroblastic colonization was observed under cylindrical ciliary epithelial cells (see FIGS. 8b, c or d for, respectively, the untreated (PLL/PGA)₄-and (PLL/PGA)₃-PLL)-coated prostheses.

### Cytokine Production

The effect of α-MSH adsorbed or embedded in a polyelectrolyte multilayer film on IL-10 secretion was measured after a given period of implantation (days 0, 3, 7, 14 and 28).

For this evaluation, PGA terminating architectures were chosen as the most favourable coating.

None of the untreated (4 rats) or treated prostheses (14 rats) induced detectable production of TNF-α, confirming the low inflammatory reaction observed in our previous histologic analyses performed with untreated prostheses and materialized by the low lymphocyte density present in the tissue surrounding the prostheses.

None of the rats (6 rats) implanted with prostheses coated with polyelectrolyte multilayers that did not include α-MSH, induced IL-10 production.

FIG. 9 shows that when adding PGA-α-MSH either on the top of the film prosthesis coating (FIGS. 9a and b, white bar: PLL/(PGA/PLL)₄/PGA-a-MSH), or embedded in prosthesis coating (FIGS. 9c and d, white bar: PLL/PGA/PLL/PGA-α-MSH/(PLL/PGA)₃), the systemic expression of IL-10 was detectable from day 3 to day 7 after implantation. None of these rats induced detectable production of TNF-α (FIG. 9a-d, black bar).

The in vivo inflammatory response to biomaterial could be followed until day 21 after implantation.

These results demonstrated that α-MSH remains, in vivo, biologically active both at the surface or embedded in the multilayer.

1. Materials useful for making support and/or replacement of living tissues, comprising microparticles of a biomaterial coated with polyelectrolyte multilayers containing one or more biologically active products, wherein the microparticles have a particle size distribution between 50-800 μm, and are fused, the porous space between contiguous particles having an average dimension of 15 to 250 μm.

2. Artificial prostheses for substitution of tissues, particularly bones and/or cartilages and/or soft tissues, based on materials according to claim 1.

3. Artificial prostheses according to claim 2, wherein the biologically active product(s) is (are) adsorbed on a polyelectrolyte multilayer film.

4. Artificial prostheses according to claim 2, wherein the biologically active product(s) is (are) embedded in a polyelectrolyte multilayer film.

5. Artificial prostheses according to claim 2, wherein the biologically active products are at different depths in the multilayer architecture.

6. Artificial prosthesis according to claim 2, wherein the biologically active products are identical or different.

7. Artificial prostheses according to claim 2, wherein the biologically active products comprise peptides, polypeptides, amino-acids derivatives, growth factors, stem cells or drugs.

8. Artificial prostheses according to claim 7, wherein the drugs are anti-inflammatory molecules or antibiotics.

9. Artificial prostheses according to claim 8, wherein the anti-inflammatory drugs are a-melanocyte-stimulating hormone (a-MSH) and its two analogues CP1 and CP2.

10. Artificial prostheses according to claim 1, wherein the polyelectrolyte multilayer films are made of polypeptides selected from the group comprising poly (L-lysine) (PLL) and poly (L-glutamic acid) (PGA).

11. Artificial prostheses according to claim 10, characterized in that the α-MSH peptide is covalently bound to PGA adsorbed on or embedded in a polyelectrolyte multi layer film (PLL/PGA)₄.

12. Artificial prostheses according to claim 2, wherein said microparticles are made of titanium or a titanium-based alloy which can contain at least one other metal chosen from among indium, tin, niobium, palladium/zirconium, tantalum, chromium, gold and silicon.

13. Artificial prostheses according to claim 2, wherein the microparticles are microspheres or microbeads.

14. Artificial prostheses according to claim 2, wherein said prostheses are intended for substitution of tracheal or laryngeal cartilages, and have sizes corresponding to mean values of trachea or larynx diameter and length, with holes at both extremities and, if necessary, a longitudinal slot.

15. Use of artificial prostheses according to claim 1 to modify chondrocyte adhesion mechanism.

16. Use according to claim 15 for a systemic anti-inflammatory IL-10 production.
